(19) United States
(12) Patent Application Publication
(10) Pub. No.: US 2025/0281560 A1
(43) Pub. Date: Sep. 11, 2025

(54) METHOD FOR PREVENTING AND/OR TREATING CANCER OR AUTOIMMUNE DISEASE WITH 3,4,2 Prime,3 Prime,4 Prime-PENTAHYDROXY-TRANS-CHALCONE

(71) Applicant: National Chung Hsing University, Taichung City (TW)

(72) Inventors: Hui-Chih Hung, Taichung City (TW); Chi-Chen Lin, Taichung City (TW); Jyh-Horng Wu, Taichung City (TW); Tung Yu Tang, Taichung City (TW)

(21) Appl. No.: 18/666,219

(22) Filed: May 16, 2024

(30) Foreign Application Priority Data
Mar. 7, 2024 (TW) 113108452

(51) Int. Cl. A61K 36/48; A61K 31/121; A61K 31/352; A61P 19/02

(52) U.S. Cl. CPC A61K 36/48; A61K 31/121; A61K 31/352; A61P 19/02; A61K 2236/39

(57) ABSTRACT

A method for preventing and/or treating cancer or an autoimmune disease with 3,4,2′,3′,4′-pentahydroxy-trans-chalcone includes administering an effective amount of 3,4,2′,3′,4′-pentahydroxy-trans-chalcone, or of a composition containing the 3,4,2′,3′,4′-pentahydroxy-trans-chalcone, to an individual to inhibit the activity of human ornithine decarboxylase and human peptidylarginine deiminase 4, thereby treating or alleviating a cancer and/or an autoimmune disease effectively.

### BACKGROUND OF THE INVENTION

### 1. Technical Field

The present invention relates to a method of treating a disease. More particularly, the invention relates to a method for preventing and/or treating cancer or an autoimmune disease with 3,4,2′,3′,4′-pentahydroxy-trans-chalcone.

### 2. Description of Related Art

Acacia confusa, also known as Formosa acacia, acacia petit feuille, Ayangile, or small Philippine acacia, is a plant of the genus Acacia of the Mimosoid clade in the family Fabaceae. Acacia confusa is an important tree species in afforestation because its root system has a firm grip on soil and because of its resistance to drought. The wood of Acacia confusa is hard, has distinct annual growth rings, and is therefore suitable for making railroad ties, furniture, and charcoal. In addition, recent studies indicate that Acacia confusa heartwood extracts have antioxidant and anti-inflammatory activity.

Human ornithine decarboxylase (ODC) is an enzyme critical to the production of polyamines in the human body. Human ornithine decarboxylase is known to be related to a variety of cancers such as breast cancer and skin cancer. Research has shown that effectively inhibiting the activity of human ornithine decarboxylase can effectively halt the growth and development of cancer cells effectively, thereby preventing or reducing the occurrence of cancer.

Human peptidylarginine deiminase 4 (PAD4) is an enzyme closely related to autoimmune diseases (such as rheumatoid arthritis) and cancer. Research has shown that immunomodulation can be achieved by inhibiting the activity of human peptidylarginine deiminase 4, thereby improving rheumatoid arthritis and potentially preventing or reducing the occurrence of cancer.

### BRIEF SUMMARY OF THE INVENTION

The primary objective of the present invention is to provide a method for preventing and/or treating cancer or an autoimmune disease with 3,4,2′,3′,4′-pentahydroxy-trans-chalcone. More specifically, administering an effective amount of 3,4,2′,3′,4′-pentahydroxy-trans-chalcone or a composition thereof disclosed herein to an individual will be able to effectively inhibit the activity of an enzyme in the individual's body that is related to a cancer or an autoimmune disease, such that through immunomodulation or by inhibiting the new growth of cancer cells, the intended effect of treating the cancer or the autoimmune disease can be achieved.

To attain the foregoing objective, the present invention discloses a method for preventing and/or treating cancer or an autoimmune disease with 3,4,2′,3′,4′-pentahydroxy-trans-chalcone. The method includes administering an effective amount of 3,4,2′,3′,4′-pentahydroxy-trans-chalcone or a composition thereof to an individual in order to treat an autoimmune disease or cancer.

The individual may be a human.

The composition may be a drug, a complementary therapy product, a nutritional supplement, or a food.

In the other embodiment of this invention, the 3,4,2′,3′,4′-pentahydroxy-trans-chalcone or a composition thereof is used as an enzyme inhibitor, which can be used to inhibit the activity of ornithine decarboxylase and/or peptidylarginine deiminase 4.

The symptoms of the autoimmune disease may include the activity of human peptidylarginine deiminase 4 being higher than a normal level.

The autoimmune disease may be rheumatoid arthritis.

The symptoms of the cancer may include the activity of ornithine decarboxylase and/or peptidylarginine deiminase 4 being higher than a normal level.

In the other embodiment of the present invention, by administering the effective amount of 3,4,2′,3′,4′-pentahydroxy-trans-chalcone or the composition thereof to the individual, it can achieve preventing and/or treating a disease which is related to the abnormal activity of human peptidylarginine deiminase 4 or ornithine decarboxylase.

In one embodiment of the present invention, 3,4,2′,3′,4′-pentahydroxy-trans-chalcone can be obtained from the Acacia confusa extract.

In one embodiment of the present invention, the composition includes the Acacia confusa extract.

Wherein, the composition further includes the following compounds: (-)-2,3-trans-3′,4′,7,8-tetrahydroxydihydroflavonol, 3,7,8,3′-tetrahydroxy-4′-methoxyflavone, 7,8,3′,4′-tetrahydroxyflavone.

Furthermore, the Acacia confusa extract is obtained by performing an extraction process on Acacia confusa using at least one extraction solvent, and the at least one extraction solvent includes a primary alcohol.

Preferably, the Acacia confusa extract is extracted from the heartwood of Acacia confusa.

Preferably, the at least one extraction solvent is methanol, ethanol, water, n-butanol, ethyl acetate, or a combination of at least two of the above.

Preferably, the Acacia confusa extract essentially includes the following compounds: (-)-2,3-trans-3′,4′,7,8-tetrahydroxydihydroflavonol, 3,7,8,3′-tetrahydroxy-4′-methoxyflavone, 7,8,3′,4′-tetrahydroxyflavone, and 3,4,2′,3′,4′-pentahydroxy-trans-chalcone.

In one embodiment of the present invention, the Acacia confusa extract is prepared by the following step a or by the following steps a and b:
Step a: immersing Acacia confusa in a first extraction solvent to carry out a first extraction treatment, and removing impurities to obtain a first extract, wherein the first extraction solvent includes a primary alcohol;
Step b: performing a second extraction treatment on the first extract using at least one second extraction solvent, and removing impurities to obtain the Acacia confusa extract, wherein the at least one second extraction solvent includes water, n-hexane, n-butanol, and ethyl acetate.

The Acacia confusa extract may be extracted from Acacia confusa using ethanol as the at least one extraction solvent.

The Acacia confusa extract may be extracted from Acacia confusa using ethanol and ethyl acetate as the at least one extraction solvent.

### BRIEF DESCRIPTION OF THE SEVERAL VIEWS OF THE DRAWINGS

FIG. 1 shows the test results of how well an Acacia confusa crude extract, an Acacia confusa water extract, an Acacia confusa n-butanol extract, and an Acacia confusa ethyl acetate extract inhibited the activity of human ornithine decarboxylase (ODC).

FIG. 2A shows the test results of how well an Acacia confusa crude extract, an Acacia confusa n-butanol extract, and an Acacia confusa ethyl acetate extract inhibited the activity of human peptidylarginine deiminase 4 (PAD4).

FIG. 2B shows the test results of how well an Acacia confusa water extract inhibited the activity of human PAD4.

FIG. 3 shows the analysis result of the clinical score of rheumatoid arthritis of each group of mice in an animal test.

FIG. 4 shows the body weight of each group of mice during the animal test.

FIG. 5 shows H&E stained sections of the knee joint tissue of each group of mice.

FIG. 6 shows the score of inflammatory cell infiltration of the knee joints of each group of mice.

FIG. 7 shows the score of synovial proliferation of the knee joints of each group of mice.

FIG. 8 shows the score of cartilage erosion of the knee joints of each group of mice.

FIG. 9 shows the result of inhibiting activity of ornithine decarboxylase of Melanoxetin (3,7,8,3′,4′-pentahydroxyflavone), Okanin (3,4,2′,3′,4′-pentahydroxy-trans-chalcone) and Transilitin (7,8,3′,4′-tetrahydroxyflavone).

FIG. 10 shows the result of inhibiting the activity of peptidylarginine deiminase 4 (PAD4) of Melanoxetin, Okanin and Transilitin.

### DETAILED DESCRIPTION OF THE INVENTION

The present invention discloses a method for preventing and/or treating cancer or an autoimmune disease with 3,4,2′,3′,4′-pentahydroxy-trans-chalcone. The method includes administering an effective amount of 3,4,2′,3′,4′-pentahydroxy-trans-chalcone or a composition thereof to an individual to effectively treat or alleviate a cancer and/or an autoimmune disease, wherein the 3,4,2′,3′,4′-pentahydroxy-trans-chalcone has the ability of inhibiting both human ornithine decarboxylase and human peptidylarginine deiminase 4.

In one embodiment of the present invention, the cancer is breast cancer, skin cancer, or other malignant tumors.

In one embodiment of the present invention, the autoimmune disease is rheumatoid arthritis. That is to say, administering an effective amount of 3,4,2′,3′,4′-pentahydroxy-trans-chalcone disclosed herein to a patient with rheumatoid arthritis or its symptoms can effectively improve or treat rheumatoid arthritis or its symptoms, wherein the symptoms of rheumatoid arthritis include joint swelling, joint inflammation, cartilage wear, and so on.

Furthermore, 3,4,2′,3′,4′-pentahydroxy-trans-chalcone can be isolated from a plant extract or prepared by artificial synthesis, wherein the method of artificial synthesis is well known by the skilled person in this field.

For example, the plant extract is Acacia confusa extract. It means that 3,4,2′,3′,4′-pentahydroxy-trans-chalcone can be prepared from a Acacia confusa extract.

In another embodiment of this invention, the composition includes the Acacia confusa extract.

In the other embodiment of the invention, the composition further inlcudes at least one compound from the Acacia confusa extract, such as (-)-2,3-trans-3′,4′,7,8-tetrahydroxydihydroflavonol, 3,7,8,3′-tetrahydroxy-4′-methoxyflavone, 7,8,3′,4′-tetrahydroxyflavone.

The Acacia confusa extract is obtained by performing an extraction process on Acacia confusa heartwood using at least one predetermined extraction solvent, wherein the at least one extraction solvent may be methanol, ethanol, water, n-butanol, ethyl acetate, or a combination of at least two of the above. And the Acacia confusa extract at least includes the following compounds: (-)-2,3-trans-3′,4′,7,8-tetrahydroxydihydroflavonol, 3,7,8,3′-tetrahydroxy-4′-methoxyflavone, 7,8,3′,4′-tetrahydroxyflavone, and 3,4,2′,3′,4′-pentahydroxy-trans-chalcone.

In one embodiment of the present invention, the Acacia confusa extract is extracted from Acacia confusa heartwood using an extraction solvent that is a primary alcohol, such as methanol or ethanol.

More specifically, the Acacia confusa heartwood is immersed in the extraction solvent for a predetermined period of time before a filtering and separation treatment is performed. The predetermined period of time is 5-10 days.

In another embodiment of the present invention, the Acacia confusa extract disclosed herein is extracted from Acacia confusa heartwood through a two-stage extraction process that includes a first-stage extraction treatment and a second-stage extraction treatment.

In the first-stage extraction treatment, a first extraction solvent is used to extract a first extract from the Acacia confusa heartwood. The first extract is also referred to as an Acacia confusa crude extract. The first extraction solvent is composed of a primary alcohol and water, wherein the primary alcohol may be methanol or ethanol.

In the second-stage extraction treatment, at least one second extraction solvent is used to extract and separate a second extract from the first extract, and the second extract is the Acacia confusa extract disclosed herein. The at least one second extraction solvent may be n-hexane, water, n-butanol, ethyl acetate, or a combination of at least two of the above. The at least one second extraction solvent can be replaced by a solvent having the same properties and features as those of any of the foregoing.

In the first-stage extraction treatment, the Acacia confusa heartwood is immersed in the first extraction solvent for at least once, with each immersion lasting for about 5-10 days, such as 5 days, 6 days, 7 days, 8 days, 9 days, or 10 days. After the at least one immersion, impurities are removed from the extract solution to obtain a filtrate. The filtrate is then subjected to a concentration treatment and a drying treatment to obtain the first extract.

Methods that can be used to remove impurities from the extract solution include filtering the extract solution with filter paper, allowing the extract solution to rest in order for precipitation to take place and then removing the precipitate, and so on.

The concentration treatment is used to reduce the water or other liquid in the filtrate. Common methods for carrying out the concentration treatment include concentration by evaporation, concentration by freezing, thin-film concentration, reduced-pressure concentration, and so on.

The drying treatment is used to remove, or change the phase of, most of the water in the filtrate. Common methods for carrying out the drying treatment include freeze drying, hot-air drying, and so on.

In the second-stage extraction treatment, the at least one second extraction solvent may be a single extraction solvent. Alternatively, different extraction solvents may be sequentially used in the second-stage extraction treatment in order to increase the active-ingredient contents of the Acacia confusa extract. For example, water, n-hexane, n-butanol, and ethyl acetate may be added one after another during the second-stage extraction treatment to produce a plurality of layers each corresponding to one of the solvents, and the layers are sequentially separated to obtain an Acacia confusa water extract, an Acacia confusa n-butanol extract, and an Acacia confusa ethyl acetate extract.

Each second extract thus obtained may be named according to the extraction solvent in the corresponding layer resulting from the second-stage extraction treatment. When water is used as the only second extraction solvent in the second-stage extraction treatment, the resulting second extract may be named an Acacia confusa water extract. When water, n-butanol, and ethyl acetate are sequentially used in the second-stage extraction treatment, the extract obtained from each layer may be named after the extraction solvent in the layer, i.e., the extraction solvent in which the corresponding fraction is dissolved. For example, the water-soluble fraction is named an Acacia confusa water extract, the n-butanol-soluble fraction is named an Acacia confusa n-butanol extract, and the ethyl acetate-soluble fraction is named an Acacia confusa ethyl acetate extract.

In some embodiments of the present invention, the Acacia confusa extract disclosed herein may be the first extract or the second extract obtained by the foregoing extraction method, and more specifically, the Acacia confusa extract contains an ethanol-soluble fraction or an ethyl acetate-soluble fraction.

Continued from the previous paragraph, the Acacia confusa extract has the following properties: solubility in ethanol and ethyl acetate and insolubility in n-butanol.

Continued from the previous paragraph, the ethanol-soluble faction essentially includes the following ingredients: (-)-2,3-trans-3′,4′,7,8-tetrahydroxydihydroflavonol, (-)-2,3-cis-3,4-cis-4′-methoxy-3,3′,4,7,8-penta hydroxyflavan, 3,7,8,3′-tetrahydroxy-4′-methoxyflavone, 7,8,3′,4′-tetrahydroxyflavone, and 3,4,2′,3′,4′-pentahydroxy-trans-chalcone.

Continued from the previous paragraph, the ethyl acetate-soluble faction essentially includes the following ingredients: 3,4-dihydroxybenzoic acid, (-)-2,3-trans-3′,4′,7,8-tetrahydroxydihydroflavonol, 3-hydroxy-4-methoxybenzoic acid, 7,8,3′,4′-tetrahydroxyflavanone, 3,4-dihydroxybenzoic acid methyl ester, 3,7,8,3′,4′-pentahydroxyflavone, 7,8,3′,4′-tetrahydroxy-3-methoxyflavone, 3,4-dihydroxybenzoic acid ethyl ester, 3,4,2′,3′,4′-pentahydroxy-trans-chalcone, 3,7,8,3′-tetrahydroxy-4′-methoxyflavone, 7,8,3′,4′-tetrahydroxy-4-methoxyflavan-3-ol, 7,8,3′,4′-tetrahydroxyflavone, 7,8,3′-trihydroxy-3,4′-dimethoxyflavone, 7,3′,4′-trihydroxyflavone, and 7,3′,4′-trihydroxy-3-methoxyflavone.

The term “heartwood” refers to wood that is in the central portion of a tree trunk, that is aged xylem without living cells, and that lies between the pith and the sapwood.

The terms “extraction process” and “extraction” refer to a process that, by taking advantage of the different solubility of each constituent substance in a mixture, can separate a target substance, or a distinctive portion containing the target substance, from the mixture.

The term “composition” refers to a substance that contains the 3,4,2′,3′,4′-pentahydroxy-trans-chalcone disclosed herein as a main active ingredient, that can be prepared in different dosage forms depending on such factors as the target users and the method of use, and that may be added with an excipient, a vehicle, and/or other ingredients that are pharmaceutically acceptable or are acceptable in the food industry. For example, the composition may be a pharmaceutical, a nutritional supplement, a food, a preparation having a therapeutic effect or an enzyme inhibitor.

The term “autoimmune disease” refers to a disease caused by an abnormal immune response in the human body that attacks normal cells. Some notable examples of autoimmune diseases are rheumatoid arthritis, lupus erythematosus, and thyroid disease.

The term “effective amount” refers to an amount in which an extract is administered and that can alleviate one or more symptoms of the disease to be treated, reduce the discomfort of one or more symptoms of the disease, or slow down the progression of the disease.

The term “treating” refers to reversing, alleviating, or inhibiting the progression, or one or more symptoms, of the disease to which the term is applied or preventing the occurrence of the disease or complications thereof. The term “treatment” refers to a therapeutic action of “treating” as defined herein.

The term “enzyme inhibitor” refers to a substance that, when administered to an individual in an effective amount, can effectively inhibit the activity of an enzyme in the individual's body and thereby prevent or treat a disease caused by abnormal activity of the enzyme. In the present invention, for example, the enzyme inhibitor is a human ornithine decarboxylase inhibitor or a human peptidylarginine deiminase 4 inhibitor.

The term “human ornithine decarboxylase inhibitor” refers to a substance that, when administered to an individual in an effective amount, can effectively inhibit the activity of the human ornithine decarboxylase in the individual's body, thereby hindering the new growth of cancer cells and consequently treating, or slowing down the progression of, cancer.

The term “human peptidylarginine deiminase 4 inhibitor” refers to a substance that, when administered to an individual in an effective amount, can effectively inhibit the activity of the human peptidylarginine deiminase 4 in the individual's body so as to treat cancer or an autoimmune disease by way of immunomodulation or anti-cancer activity.

The technical features of the present invention and their effects are detailed below with reference to some examples and the accompanying drawings.

### Example 1: Preparation of Acacia confusa Extracts

Room-temperature extraction was carried out by immersing 1649 g of Acacia confusa heartwood in 20 L of 70% ethanol for at least once, with each immersion lasting for 7 days. The extract solution was filtered through filter paper by a suction filtration process to remove impurities, and the filtrate was concentrated by a rotary vacuum evaporator and subsequently moved into a freeze dryer for freeze drying. The result was an Acacia confusa crude extract.

The Acacia confusa crude extract was added, and thoroughly mixed, with distilled water, which was 15 times (w/v) as much as the crude extract. After that, fractional distillation was performed sequentially with n-hexane, n-butanol, and ethyl acetate in order to obtain different fractions. More specifically, the aqueous solution of the Acacia confusa crude extract was added with the same amount of n-hexane, and an n-hexane-soluble fraction and a water layer were collected through a separatory funnel. Partition chromatography was then carried out by adding n-butanol (BuOH) and ethyl acetate sequentially into the Acacia confusa water layer. An n-hexane-soluble fraction, an ethyl acetate-soluble fraction, an n-butanol-soluble fraction, a water-soluble fraction, and a water-insoluble fraction were collected as a result. The water-soluble fraction was an Acacia confusa water extract, the n-butanol-soluble fraction was an Acacia confusa n-butanol extract, and the ethyl acetate-soluble fraction was an Acacia confusa ethyl acetate extract.

### Example 2: Test on Inhibition of the Activity of Human Ornithine Decarboxylase

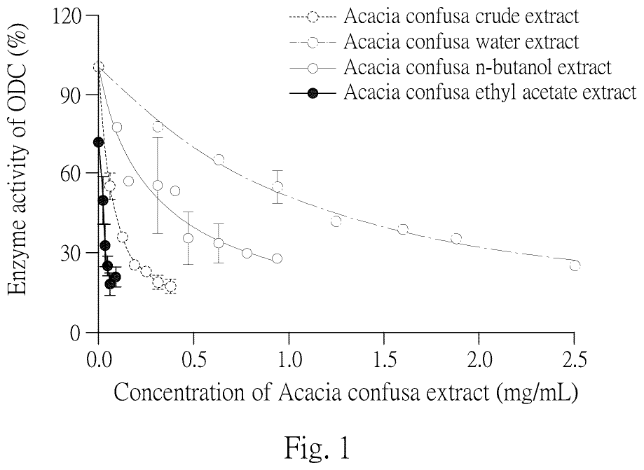

In this example, inhibition of the activity of human ornithine decarboxylase (ODC) was tested by continuous spectrophotometry. The standard ODC spectrophotometric reaction composition contained 30 mM Tris-HCl (pH 7.8), 10 mM ornithine, and 0.2 mM pyridoxal 5′-phosphate (PLP), together with 12.5 mM phosphoenolpyruvate, 0.4 unit/mL phosphoenolpyruvate carboxykinase of microbes, 4.1 unit/mL malate dehydrogenase (malate dehydrogenase of mammals), and 0.6 mM NADH analog, with the final volume being 0.5 mL. The test mixture was added with a predetermined amount of an Acacia confusa extract sample as an inhibitor and then added with an appropriate amount of ODC to start the reaction. Once the enzyme ODC was added into the reaction mixture, the decrease in absorbance at 405 nm was continuously tracked with Lambda 25 UV-visible spectrophotometer (PerkinElmer Life Sciences). By this test method, 1 mole of CO₂ was generated under the test conditions, and 1 mole of NADH analog was oxidized. The unit of the enzyme ODC was defined as the amount of the enzyme that is required to catalyze the generation of 1 mole of NAD per minute. For the calculation, 2410 M⁻¹cm⁻¹ was used as the molar absorptivity of the NADH analog. The test results are shown in FIG. 1 and Table 1.

TABLE 1
IC₅₀ values of various Acacia confusa extracts

| Sample | IC₅₀ value (μg/mL) |
|---|---|
| Acacia confusa crude extract | 72.6 |
| Acacia confusa water extract | 1044 |
| Acacia confusa n-butanol extract | 322.9 |
| Acacia confusa ethyl acetate extract | 19.2 |

It can be known from the results in FIG. 1 and Table 1 that each of the Acacia confusa crude extract, the Acacia confusa water extract, the Acacia confusa n-butanol extract, and the Acacia confusa ethyl acetate extract was effective in inhibiting human ornithine decarboxylase (ODC); that the Acacia confusa ethyl acetate extract had the best inhibiting effect, with the corresponding IC₅₀ value being 19.2 μg/mL; and that the Acacia confusa crude extract had the second best inhibiting effect, with the corresponding IC₅₀ value being 72.6 μg/mL.

### Example 3: Test on Inhibition of the Activity of Human Peptidylarginine Deiminase 4

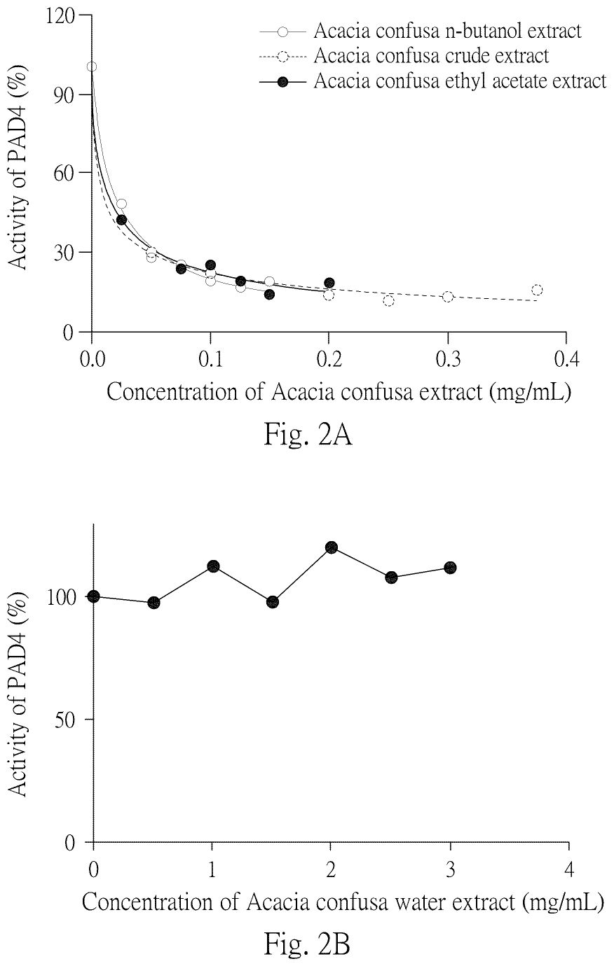

In this example, inhibition of the activity of human peptidylarginine deiminase 4 (PAD4) was tested by continuous spectrophotometry. The standard PAD4 spectrophotometric reaction mixture contained 10 mM BAEE (benzoyl-L-arginine ethyl ester), 10 mM CaCl₂), 2.5 mM dithiothreitol (DTT), 8.5 mM α-ketoglutarate (α-KG), 0.22 mM NADH, and 3 U glutamate dehydrogenase (GDH). 100 mM Tris-HCl (pH 7.5) was added into a 1-mL colorimetric tube, followed by a predetermined amount of an Acacia confusa extract sample as an inhibitor. The temperature was controlled at 37° C. An appropriate amount of PAD4 was then added into the test mixture to start the reaction. Once the enzyme PAD4 was added into the reaction mixture, the decrease in absorbance at 340 nm was continuously recorded with a spectrophotometer (Beckman DU 7500). The unit of the enzyme PAD4 was defined as the amount reduced in order to catalyze the conversion of 1 μmol of NADH to NAD⁺ per minute. For the calculation of enzyme activity, 6220 M⁻¹cm⁻¹ was used as the molar absorptivity of the NADH. The test results are shown in FIG. 2 and Table 2.

TABLE 2
IC₅₀ values of various Acacia confusa extracts

| Sample | IC₅₀ value (μg/mL) |
|---|---|
| Acacia confusa crude extract | 11 ± 4 |
| Acacia confusa n-butanol extract | 21 ± 3 |
| Acacia confusa ethyl acetate extract | 16 ± 3 |

It can be known from FIG. 2 and Table 2 that each of the Acacia confusa crude extract, the Acacia confusa n-butanol extract, and the Acacia confusa ethyl acetate extract was effective in inhibiting PAD4; that the Acacia confusa crude extract and the Acacia confusa ethyl acetate extract had equivalent effects when it comes to inhibiting the activity of PAD4, with their respective IC₅₀ values being 11 μg/mL and 16 μg/mL; and that the Acacia confusa water extract was not effective in inhibiting PAD4.

### Example 4: Animal Test

In this example, type II collagen-induced arthritis (CIA) mouse models were used as the subjects of the test. More specifically, the mice were divided into the following 4 groups:
Group 1: normal mice;
Group 2: CIA mouse models, without any drug administered;
Group 3: CIA mouse models, with the Acacia confusa ethyl acetate extract administered daily at 100 mg/kg; and
Group 4: CIA mouse models, with the Acacia confusa crude extract administered daily at 100 mg/kg.

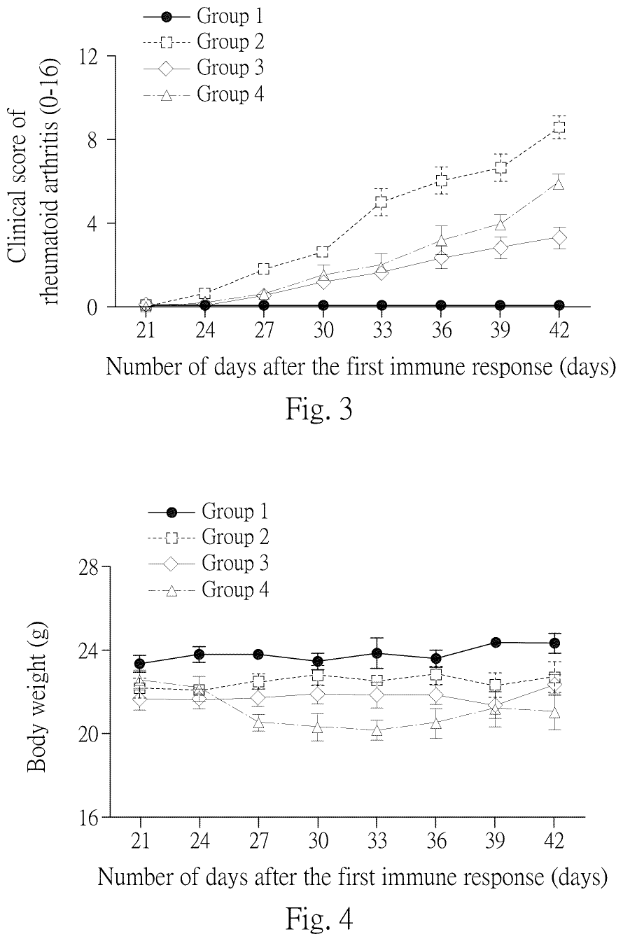

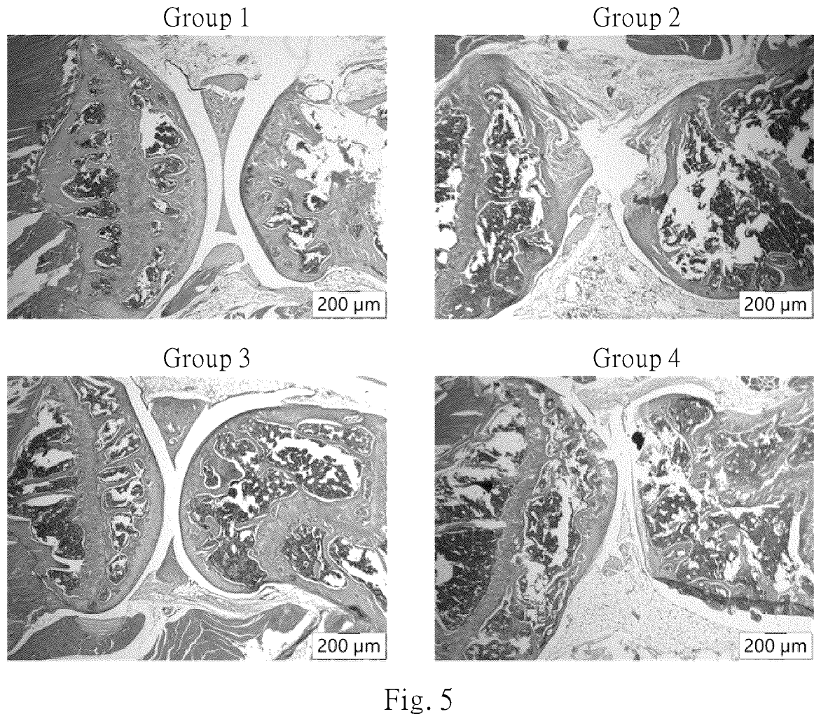

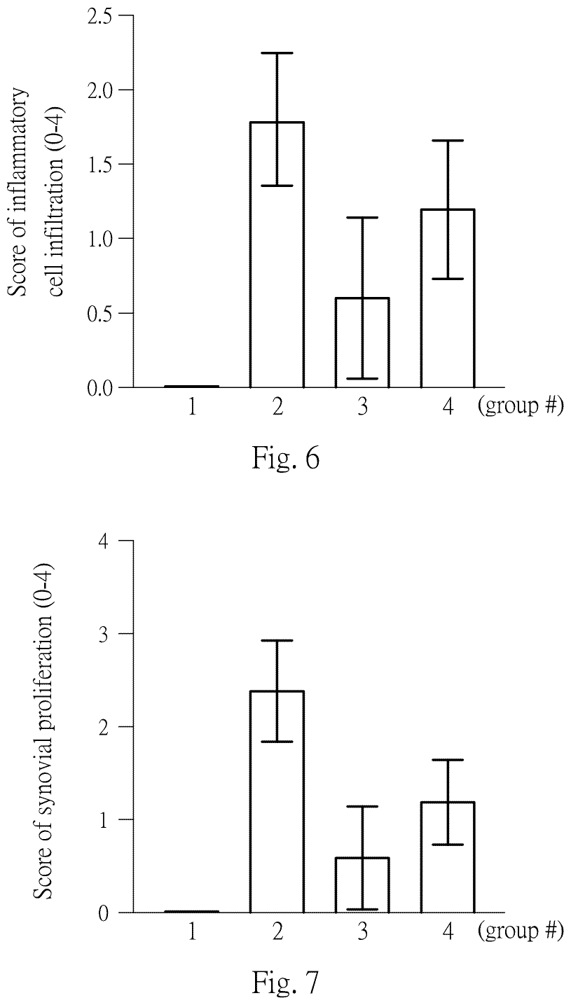

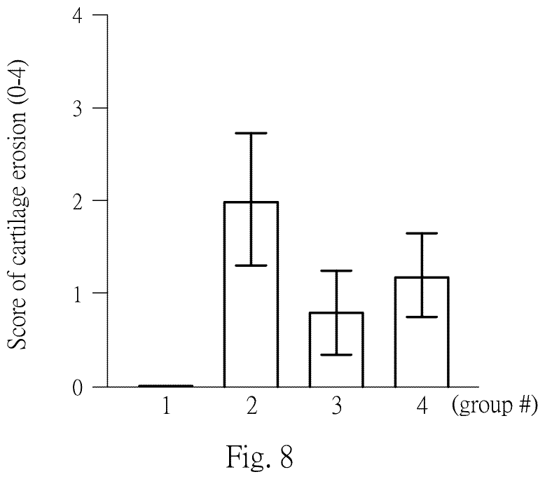

During the test, which lasted for a total of 6 weeks, the body weight of each group of mice was measured regularly, and the clinical score of rheumatoid arthritis (RA) of each group of mice was regularly evaluated after the injection of CII antigen. The results are shown in FIG. 3 and FIG. 4. After the test, the knee joints of each group of mice were collected, stained with H&E, and subjected to histopathological scoring. The scoring results are shown in FIG. 5 to FIG. 8.

It can be known from the results in FIG. 4 that there was no significant difference in body weight among the different groups of mice, and from this it can be inferred that the Acacia confusa crude extract and the Acacia confusa ethyl acetate extract disclosed herein have no adverse effect on the body weight of an individual to whom the extracts are administered. In addition, according to the results in FIG. 3, FIG. 5 to FIG. 8, the administration of an effective amount of the Acacia confusa crude extract or the Acacia confusa ethyl acetate extract disclosed herein lowered the clinical scores of rheumatoid arthritis (RA) of group 3 and group 4 of mice, and an observation of the knee joint tissue structures reveals that symptoms such as inflammation, cartilage wear, and synovial proliferation were effectively improved or inhibited. It can therefore be inferred that the Acacia confusa crude extract or the Acacia confusa ethyl acetate extract disclosed herein can be used to treat or improve an autoimmune disease such as rheumatoid arthritis.

### Example 5: Analysis of the Acacia confusa Crude Extract

The ingredients of the Acacia confusa crude extract can be analyzed with an analysis method well known in the field to which the present invention pertains, such as Sephadex LH-20 gel permeation chromatography, PR-18 column chromatography, or high-performance liquid chromatography (HPLC).

In this example, the analysis was carried out by HPLC. The column used was an RP-18 column, and the analysis conditions were as follows. The mobile phases included 100% methanol (MeOH) as solvent A and ultrapure water as solvent B. The elution conditions were: 15%-30% A to B (linear gradient) from minute 0 to minute 20, and 30%-50% A to B (linear gradient) at a flow rate of 4 mL/min from minute 20 to minute 50. The separated compounds were identified by electron ionization-mass spectrometry (EI-MS) and nuclear magnetic resonance (NMR) spectroscopy, and the results are shown in Table 3.

TABLE 3
Ingredients of the Acacia confusa crude extract

| Compound | Content (mg) |
|---|---|
| (−)-2,3-trans-3′,4′,7,8-tetrahydroxydihydroflavonol | 18.8 |
| (−)-2,3-cis-3,4-cis-4′-methoxy- 3,3′,4,7,8-penta | 5.7 |
| hydroxyflavan | |
| 3,7,8,3′-tetrahydroxy-4′-methoxyflavone (also known | 61.1 |
| as melanoxetin) | |
| 7,8,3′,4′-tetrahydroxyflavone (also known as transilitin) | 40.8 |
| 3,4,2′,3′,4′- pentahydroxy-trans-chalcone (also | 13.8 |
| known as okanin) | |

### Example 6: Analysis of the Ingredients of the Acacia confusa Ethyl Acetate Extract

The ingredients of the Acacia confusa ethyl acetate extract were analyzed by RP-18 column chromatography. The column temperature was 30° C. The mobile phase system was a mixture of two liquids mixed in a predetermined ratio, with liquid A being water, and liquid B being methanol. The sample injection amount was 500 μL. The flow rate was 10 mL/min. The detector performed multiple-wavelength detection. The ingredients of the Acacia confusa ethyl acetate extract are shown in Table 4.

TABLE 4
Ingredients of the Acacia confusa ethyl acetate extract
Compound

3,4-dihydroxybenzoic acid
(−)-2,3-trans-3′,4′,7,8-tetrahydroxydihydroflavonol
3-hydroxy-4-methoxybenzoic acid
7,8,3′,4′-tetrahydroxyflavanone
3,4-dihydroxybenzoic acid methyl ester
3,7,8,3′,4′-pentahydroxyflavone
7,8,3′,4′-tetrahydroxy-3-methoxyflavone
3,4-dihydroxybenzoic acid ethyl ester
3,4,2′,3′,4′- pentahydroxy-trans-chalcone
3,7,8,3′ -tetrahydroxy-4′-methoxyflavone
7,8,3′,4′-tetrahydroxy-4-methoxyflavan-3-ol
7,8,3′,4′-tetrahydroxyflavone
7,8,3′-trihydroxy-3,4′-dimethoxyflavone
7,3′,4′-trihydroxyflavone
7,3′,4′-trihydroxy-3-methoxyflavone

A comparison between Table 3 and Table 4 shows that (-)-2,3-trans-3′,4′,7,8-tetrahydroxydihydroflavonol, 3,7,8,3′-tetrahydroxy-4′-methoxyflavone, 7,8,3′,4′-tetrahydroxyflavone, and 3,4,2′,3′,4′-pentahydroxy-trans-chalcone were found in both the Acacia confusa crude extract and the Acacia confusa ethyl acetate extract, and it can be known from the results of example 2 and example 3 that both the Acacia confusa crude extract and the Acacia confusa ethyl acetate extract had the activity of inhibiting ornithine decarboxylase and/or peptidylarginine deiminase 4. Therefore, it can be reasonably inferred that in Acacia confusa extract, the main active ingredients for inhibiting ornithine decarboxylase and/or peptidylarginine deiminase 4 include (-)-2,3-trans-3′,4′,7,8-tetrahydroxydihydroflavonol, 3,7,8,3′-tetrahydroxy-4′-methoxyflavone, 7,8,3′,4′-tetrahydroxyflavone, and 3,4,2′,3′,4′-pentahydroxy-trans-chalcone.

Moreover, it can be known from the results of example 2 and example 3 that both the Acacia confusa crude extract and the Acacia confusa ethyl acetate extract had the activity of inhibiting human ornithine decarboxylase and human peptidylarginine deiminase 4, and that such activity of the Acacia confusa ethyl acetate extract was higher than that of the Acacia confusa crude extract. This indicates that the mixture obtained by performing the two-stage (ethanol and ethyl acetate) extraction process on Acacia confusa heartwood had had those non-active ingredients in the mixture extracted with ethanol alone removed through the two-stage extraction process. That is to say, the two-stage extraction process can enhance the enzyme-inhibiting activity of an Acacia confusa extract.

According to the foregoing results, the Acacia confusa extract disclosed herein has the activity of inhibiting human ornithine decarboxylase and human peptidylarginine deiminase 4 and can therefore be used to effectively treat or improve cancer or an autoimmune disease such as rheumatoid arthritis or its symptoms (e.g., a swollen joint or cartilage damage), and the main active ingredients of the Acacia confusa extract include (-)-2,3-trans-3′,4′,7,8-tetrahydroxydihydroflavonol, 3,7,8,3′-tetrahydroxy-4′-methoxyflavone, 7,8,3′,4′-tetrahydroxyflavone, and 3,4,2′,3′,4′-pentahydroxy-trans-chalcone.

### Example 7: Human Ornithine Decarboxylase Inhibitory Activity Test

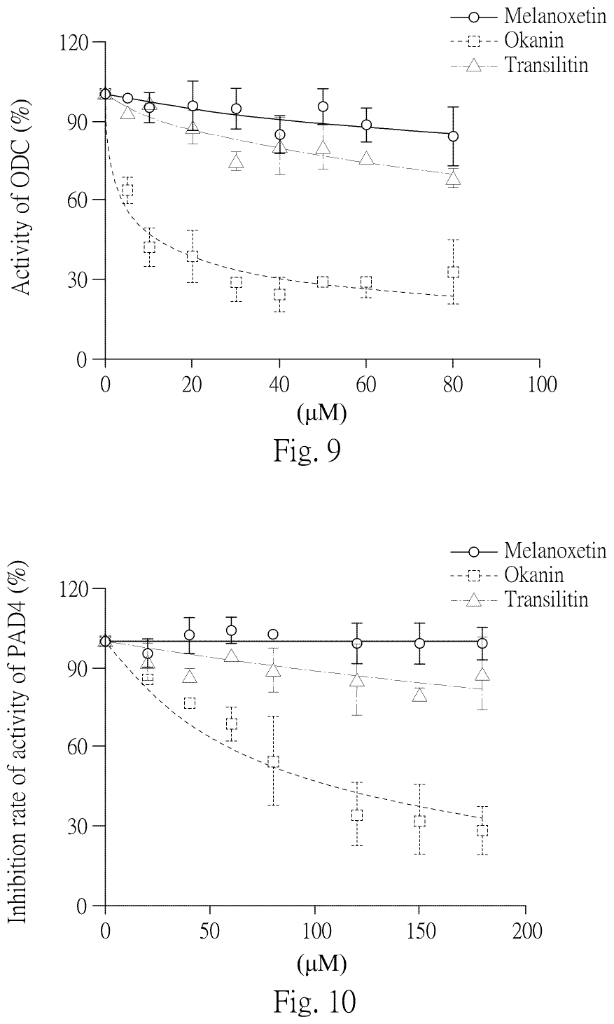

Refer to the content of Example 2 to detect the inhibiting activity of ornithine decarboxylase of components of the Acacia confusa ethyl acetate extract: Melanoxetin (3,7,8,3′,4′-pentahydroxyflavone), Okanin (3,4,2′,3′,4′-pentahydroxy-trans-chalcone) and Transilitin (7,8,3′,4′-tetrahydroxyflavone), the results are shown in FIG. 9.

From the results in FIG. 9, it can be seen that Okanin has the activity to inhibit human ornithine decarboxylase, wherein IC50 is 8.2±2.1 μM.

### Example 8: Human Peptidylarginine Deiminase 4 (PAD4) Inhibitory Activity Test

Referring to the content of Example 3 to detect the inhibiting activity of peptidylarginine deiminase 4 (PAD4) of components of the Acacia confusa ethyl acetate extract: Melanoxetin, Okanin and Transilitin, the results are shown in FIG. 10.

From the results in FIG. 10, it can be seen that Okanin has the activity of inhibiting human peptidylarginine deiminase 4 (PAD4), wherein IC50 is 89.2±8.3 μM.

Comprehensive results of Examples 7 and 8 confirmed that Okanin (3,4,2′,3′,4′-pentahydroxy trans-chalcone) has the ability to simultaneously inhibit human ornithine decarboxylase and human peptidylarginine deiminase 4 (PAD4), so that Okanin can be used as a main active ingredient in the treatment or prevention of cancer or autoimmune diseases. In other words, the Okanin or the composition thereof disclosed in the present invention is an enzyme inhibitor that can be used to effectively inhibit the activity of ornithine decarboxylase or/and peptidylarginine deiminase 4 to achieve the effect of treating or preventing cancer or autoimmune diseases.

What is claimed is:

1. A method for preventing and/or treating cancer or an autoimmune disease with 3,4,2′,3′,4′-pentahydroxy-trans-chalcone, comprising: administering an effective amount of 3,4,2′,3′,4′-pentahydroxy-trans-chalcone or a composition thereof to an individual, through inhibiting activity of ornithine decarboxylase and/or peptidylarginine deiminase 4 in the individual's body for preventing and/or treating cancer or an autoimmune disease.

2. The method for preventing and/or treating cancer or an autoimmune disease with 3,4,2′,3′,4′-pentahydroxy-trans-chalcone as claimed in claim 1, wherein the composition further comprises at least one compound of the following compounds: (-)-2,3-trans-3′,4′,7,8-tetrahydroxydihydroflavonol, 3,7,8,3′-tetrahydroxy-4′-methoxyflavone, and 7,8,3′,4′-tetrahydroxyflavone.

3. The method for preventing and/or treating cancer or an autoimmune disease with 3,4,2′,3′,4′-pentahydroxy-trans-chalcone as claimed in claim 1, wherein the 3,4,2′,3′,4′-pentahydroxy-trans-chalcone is prepared from an Acacia confusa extract.

4. The method for preventing and/or treating cancer or an autoimmune disease with 3,4,2′,3′,4′-pentahydroxy-trans-chalcone as claimed in claim 1, wherein the composition comprises an Acacia confusa extract.

5. The method for preventing and/or treating cancer or an autoimmune disease with 3,4,2′,3′,4′-pentahydroxy-trans-chalcone as claimed in claim 3, wherein the Acacia confusa extract is obtained by performing an extraction process on Acacia confusa using at least one extraction solvent, and the at least one extraction solvent comprises a primary alcohol.

6. The method for preventing and/or treating cancer or an autoimmune disease with 3,4,2′,3′,4′-pentahydroxy-trans-chalcone as claimed in claim 4, wherein the Acacia confusa extract is obtained by performing an extraction process on Acacia confusa using at least one extraction solvent, and the at least one extraction solvent comprises a primary alcohol.

7. The method for preventing and/or treating cancer or an autoimmune disease with 3,4,2′,3′,4′-pentahydroxy-trans-chalcone as claimed in claim 5, wherein the Acacia confusa extract is extracted from a heartwood of the Acacia confusa.

8. The method for preventing and/or treating cancer or an autoimmune disease with 3,4,2′,3′,4′-pentahydroxy-trans-chalcone as claimed in claim 6, wherein the Acacia confusa extract is extracted from a heartwood of the Acacia confusa.

9. The method for preventing and/or treating cancer or an autoimmune disease with 3,4,2′,3′,4′-pentahydroxy-trans-chalcone as claimed in claim 5, wherein the at least one extraction solvent is one, or a combination of at least two, of methanol, ethanol, water, n-butanol, and ethyl acetate.

10. The method for preventing and/or treating cancer or an autoimmune disease with 3,4,2′,3′,4′-pentahydroxy-trans-chalcone as claimed in claim 6, wherein the at least one extraction solvent is one, or a combination of at least two, of methanol, ethanol, water, n-butanol, and ethyl acetate.

11. The method for preventing and/or treating cancer or an autoimmune disease with 3,4,2′,3′,4′-pentahydroxy-trans-chalcone as claimed in claim 5, wherein the extraction process is a two-stage extraction treatment comprising: a first-stage extraction treatment using ethanol as a first extraction solvent; and a second-stage extraction treatment using a second extraction solvent comprising ethyl acetate.

12. The method for preventing and/or treating cancer or an autoimmune disease with 3,4,2′,3′,4′-pentahydroxy-trans-chalcone as claimed in claim 6, wherein the extraction process is a two-stage extraction treatment comprising: a first-stage extraction treatment using ethanol as a first extraction solvent; and a second-stage extraction treatment using a second extraction solvent comprising ethyl acetate.

13. The method for preventing and/or treating cancer or an autoimmune disease with 3,4,2′,3′,4′-pentahydroxy-trans-chalcone as claimed in claim 1, wherein the individual is a human.

14. The method for preventing and/or treating cancer or an autoimmune disease with 3,4,2′,3′,4′-pentahydroxy-trans-chalcone as claimed in claim 13, wherein the individual has an autoimmune disease.

15. The method for preventing and/or treating cancer or an autoimmune disease with 3,4,2′,3′,4′-pentahydroxy-trans-chalcone as claimed in claim 14, wherein the autoimmune disease is rheumatoid arthritis.

16. The method for preventing and/or treating cancer or an autoimmune disease with 3,4,2′,3′,4′-pentahydroxy-trans-chalcone as claimed in claim 1, wherein the individual has a cancer.

17. The method for preventing and/or treating cancer or an autoimmune disease with 3,4,2′,3′,4′-pentahydroxy-trans-chalcone as claimed in claim 1, wherein the composition comprises the following compounds: (-)-2,3-trans-3′,4′,7,8-tetrahydroxydihydroflavonol, 3,7,8,3′-tetrahydroxy-4′-methoxyflavone, 7,8,3′,4′-tetrahydroxyflavone, and 3,4,2′,3′,4′-pentahydroxy-trans-chalcone.
